(19) United States
(12) Patent Application Publication
(10) Pub. No.: US 2026/0027277 A1
(43) Pub. Date: Jan. 29, 2026

(54) EXTRACELLULAR VESICLE DEPLETED BLOOD FRACTIONS

(71) Applicants: GRIFOLS WORLDWIDE OPERATIONS LIMITED, DUBLIN (IE); FUNDACIO PRIVADA INSTITUT D'INVESTIGACIO ONCOLOGICA DE VALL HEBRON, BARCELONA (ES)

(72) Inventors: Antonio Manuel PAEZ REGADERA, BARCELONA (ES); Alba PEREZ PERARNAU, BARCELONA (ES); Isabel BRAVO CAMISON, BARCELONA (ES); Josep TABERNERO CATURLA, BARCELONA (ES); Elena ELEZ FERNANDEZ, BARCELONA (ES); Josep VILLANUEVA CARDUS, BARCELONA (ES)

(21) Appl. No.: 18/854,528

(22) PCT Filed: Apr. 5, 2023
(86) PCT No.: PCT/EP2023/058987

§ 371 Date: Oct. 5, 2024

(30) Foreign Application Priority Data
Apr. 6, 2022 (EP) 22382329.5

(51) Int. Cl. A61M 1/34; A61K 35/16; A61P 35/00

(52) U.S. Cl. CPC A61M 1/3496; A61K 35/16; A61P 35/00; A61M 2202/0415

(57) ABSTRACT

Disclosed herein are human blood fractions depleted of disease-causing extracellular vesicles, prepared by plasma exchange, that may find use in the treatment of a cancer. In particular, the depleted blood fractions may find use in treating KRAS mutant cancers.

### FIELD OF THE INVENTION

The present invention provides for methods of decreasing extracellular vesicle load in patient plasma, and methods of treating pathologies in which said extracellular vesicles are implicated.

### BACKGROUND

Extracellular vesicles are lipid bilayer-delimited particles that are released from cells into the extracellular space by budding from the plasma membrane, or alternatively by invagination of the endosomal membrane and maturation into a multivesicular body that fuses with the plasma membrane so as to release its contents. Extracellular vesicles are thought to provide a means of intercellular communication and of transmission of macromolecules between cells.

Extracellular vesicles have been implicated as contributing factors in the development of several diseases owing to their role in the delivery of proteins, lipids, mRNA, miRNA and DNA from one cell to another. Packaging of extracellular vesicles appears to be indiscriminate, as such extracellular vesicles can provide for the delivery of both “good” and ‘bad’ cargo to their target cells. Extracellular vesicles have been reported to contain numerous disease-associated cargos, for example:
miRNAs in the case of cancer, and
neurodegenerative-associated peptides, such as AP, tau, prions, and alpha-synuclein.

Whilst knowledge about extracellular vesicles is growing, the means by which disease-associated factors spread between cells remains poorly understood even though extracellular vesicles are implicated in such processes/transmission. Moreover, a thorough understanding of the mechanism whereby particular cargos [proteins, RNAs, etc.] are sorted into particular extracellular vesicles remains elusive. Consequently, traditional pharmaceutical targeting of extracellular vesicles continues to be challenging owing to structural non-homogeneity in the various different types of extracellular vesicles, and the aforementioned knowledge gaps around the molecular mechanisms by which extracellular vesicles are packaged and spread.

As such, there remains a need for therapies that can consistently and reproducibly reduce “bad” extracellular vesicle loads in patients thereby providing a reliable means to down-regulate the spread of extracellular vesicles and the consequent pathologies/diseases associated therewith.

### DESCRIPTION OF THE INVENTION

The words “comprises/comprising” and the words “having/including” when used herein with reference to the present invention are used to specify the presence of stated features, integers, steps or components but do not preclude the presence or addition of one or more other features, integers, steps, components or groups thereof.

It should be appreciated by those skilled in the art that the specific embodiments disclosed herein should not be read in isolation, and that the present specification intends for the disclosed embodiments to be read in combination with one another as opposed to individually. As such, each embodiment may serve as a basis for modifying or limiting other embodiments disclosed herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “10 to 100” should be interpreted to include not only the explicitly recited values of 10 to 100, but also include individual value and sub-ranges within the indicated range. Thus, included in this numerical range are individual values such as 10, 11, 12, 13 . . . 97, 98, 99, 100 and sub-ranges such as from 10 to 40, from 25 to 40 and 50 to 60, etc. This same principle applies to ranges reciting only one numerical value, such as “at least 10”. Furthermore, such an interpretation should apply regardless of the breadth of the range or the characteristics being described.

### Treatment of the Invention

In a first aspect, the present invention provides for extracellular vesicle depleted human blood fractions for use in the treatment of a cancer, wherein blood obtained from a patient diagnosed with the cancer is subjected to plasma exchange so as to deplete the extracellular vesicle content prior to being re-administered to said patient.

In a second aspect, the present invention provides for use of plasma exchange for removing extracellular vesicles from a patient in need thereof, wherein
the patient is a patient diagnosed with a cancer, and
further wherein no binding agent that specifically binds the extracellular vesicles is utilised in the plasma exchange process.

By “blood fraction” it is meant any derivative of whole blood that has been processed or fractionated to alter the natural concentrations of red blood cells, white blood cells, platelets, or plasma in whole blood. In some embodiments, “blood fraction” shall be construed to mean a derivative of whole blood that has been processed to reduce or alter the plasma component alone. Suitable methods of generating blood fractions from whole blood include, without limitation, plasmapheresis.

By “depleted” the present specification means depleted of disease-causing extracellular vesicles compared to a blood sample from the patient that had not been subjected to the plasma exchange procedure. In some embodiments, depletion may equate to a greater than 90% reduction in disease-causing extracellular vesicles compared to a blood sample from the patient that had not been subjected to the plasma exchange procedure. For example, depletion may equate to a reduction greater than 80%, 70%, 60%, 50%, 40%, 30%, 20%, or 10% in disease-causing extracellular vesicles compared to a blood sample from the patient that had not been subjected to the plasma exchange procedure. Disease causing extracellular vesicles may be identified by targeting particular sets of biomarkers outlined in the Detailed Examples of the Invention and assessing the contents of the extracellular vesicles. Such techniques are within the repertoire of a person of skill in the art.

In some embodiments, prior to being treated with the extracellular vesicle depleted human blood fraction of the present invention the patient diagnosed with the cancer has been assessed to determine the presence or absence of disease-causing extracellular vesicles in their plasma. For example, in certain embodiments the patient may have tested positive for disease-causing extracellular vesicles prior to the treatment of the present invention.

In other embodiments, the patient diagnosed with the cancer may be non-responsive/refractory to the standard of care/first line pharmacological treatment (for said cancer) with small molecule drugs/biological drugs prior to treatment with the extracellular vesicle depleted human blood fraction of the present invention. The present invention envisages that the innovative treatments disclosed herein could be administered complimentarily to the standard of care treatments as opposed to the two being mutually exclusive.

As used herein, the term “plasma exchange” means a procedure in which a patient's blood is passed through a device, for example a plasmapheresis device, and the plasma component filtered by the device is removed and discarded. Red blood cells and other non-filtered blood fractions, optionally along with replacement fluid such as fresh frozen plasma or albumin, are reinfused back into the patient. Traditionally, the efficacy of plasma exchange is proportional to the plasma volume removed in relation to the patient's total plasma volume.

Advantageously, with respect to both aspects of the present invention, subjecting the patient's blood to plasma exchange without the incorporation of any specific extracellular vesicle binding agents (such as antibodies, antibody fragments, aptamers, etc.) circumvents problems associated with non-reproducible binding of structurally non-homogeneous extracellular vesicles. Moreover, obviating the need for expensive binding reagents greatly reduces the cost of any treatment for patients and health care providers. As used herein, “extracellular vesicle binding agent” shall be construed to mean materials capable of selectively binding with the extracellular vesicles over other plasma components and include antibodies, antibody fragments, other binding proteins, aptamers, and the like.

For the avoidance of any doubt, plasma exchange as referred to within this specification does not comprise the utilisation of any selective extracellular vesicle binding agents as part of the process.

By “extracellular vesicle”, the present specification means lipid bilayer-delimited particles that contain macromolecules such as proteins, lipids, nucleic acids, and combinations thereof that are released from cells into the intracellular space and have a diameter of 20-200 nm as determined by cryo-transmission electron microscopy (CRYO-TEM) analysis at −179° C. and an accelerating voltage of 200 kV. In one embodiment, the extracellular vesicles may be between 30-200 nm in diameter, 30-150 nm in diameter, 30-120 nm in diameter, 30-100 nm in diameter, 40-200 nm in diameter, 40-150 nm in diameter, 40-120 nm in diameter, or 40-100 nm in diameter. Extracellular vesicles as referred to in the present specification may be enriched in tetraspanins CD9, CD63, CD81, and combinations thereof.

The extracellular vesicles as referred to in the present specification may be 30-200 nm in diameter, 30-150 nm in diameter, 30-120 nm in diameter, or 30-100 nm in diameter as determined by cryo-transmission electron microscopy analysis [at −179° C. and an accelerating voltage of 200 kV] and may be enriched in tetraspanins CD9, CD63, CD81, and combinations thereof. For example, the extracellular vesicles as referred to in the present specification may be 40-200 nm in diameter, 40-150 nm in diameter, 40-120 nm in diameter, or 40-100 nm in diameter as determined by cryo-transmission electron microscopy analysis at −179° C. and may be enriched in tetraspanins CD9, CD63, CD81, and combinations thereof.

### Oncology

The depleted blood fractions of the present invention may be for use in the treatment of cancer. Similarly, with respect to the second aspect of the present invention, the use of plasma exchange for removing extracellular vesicles in a patient may be utilised where the patient is a patient diagnosed with cancer.

For example, the cancer may be selected from the group consisting of soft tissue sarcomas, kidney cancer, liver cancer, intestinal cancer, rectal cancer, leukaemia, lymphomas, brain cancer, oesophageal cancer, uterine cancer, cervical cancer, bone cancer, lung cancer, bladder cancer, breast cancer, laryngeal cancer, colorectal cancer, stomach cancer, ovarian cancer, pancreatic cancer, adrenal gland cancer, prostate cancer, and combinations thereof.

In one embodiment, the cancer is selected from the group consisting of colorectal cancer, pancreatic cancer, and lung cancer.

In some embodiments, the cancer is refractory to chemotherapy treatment.

In particular embodiments, the cancer is a KRAS mutant cancer. As used herein, the term “KRAS mutant cancer” shall be construed to mean any cancer in which the KRAS gene is mutated such that it is stuck in the “on” position, allowing cells to grow uncontrollably leading to multiplication of cells, cancer growth, and may ultimately cause metastases.

In certain embodiments, the cancer may be a KRAS mutant cancer refractory to chemotherapy treatment.

In one embodiment, the KRAS mutant cancer may be KRAS mutant colorectal cancer, KRAS mutant pancreatic cancer, and KRAS mutant lung cancer. In some embodiments, the KRAS mutant cancer may be a metastatic cancer. For example, the KRAS mutant cancer may be KRAS mutant metastatic colorectal cancer, KRAS mutant metastatic pancreatic cancer, and KRAS mutant metastatic lung cancer.

In certain embodiments, the depleted blood fraction of the present invention is for use in the treatment of colorectal cancer. For example, the colorectal cancer may be metastatic colorectal cancer. In other embodiments, the colorectal cancer may be refractory to chemotherapy treatment. In some embodiments, the colorectal cancer may be KRAS mutant metastatic colorectal cancer. For example, the colorectal cancer may be KRAS mutant metastatic colorectal cancer refractory to first line chemotherapy treatment.

It should be appreciated by those skilled in the art that the specific embodiments disclosed within paragraphs [0021]-[0027] should not be read in isolation, and that the present specification intends for these embodiments to be disclosed in combination with other embodiments as opposed to being disclosed individually. For example, each of the embodiments disclosed in paragraphs [0009]-[0019] is to be read as being explicitly combined with each of the embodiments in paragraphs [0020]-[0027], or any permutation of 2 or more of the embodiments disclosed therein.

### Plasma Exchange Volumes

Typically, a patient's total blood volume is calculated as per Nadler's formula (Nadler S B, Hidalao J H, Bloch T. Prediction of blood volume in normal human adults. Surgery. 1962:51(2):224-32), reproduced below:

### Patient Total Blood Volume (mL):

$$\begin{matrix}Male = ( 0. 06 \; 012 \times {H}^{3} ) / ( 1 4.6 \times W ) + 604 \\ Female = ( 0. 005 \; 835 \times {H}^{3} ) / ( 15 \times W ) + 183 \\ H = height in inches , W = weight in pounds .\end{matrix}$$

Plasma exchange procedures report the quantity of blood/plasma processed in terms of a patient's total blood volume. Processing 1 Volume equates to processing the patient's total blood volume as determined by Nadler's formula. Similarly, processing 1.5 volumes equates to processing “1.5× the patient's total blood volume” as determined by Nadler's formula. Naturally, volumes over 1 result in some of the patient's blood volume being processed more than once.

Plasma exchanges can also be reported in terms of plasma volumes, eg 1 plasma volume. Given that plasma accounts for about 55% of total blood volume the two naming systems are interrelated and ultimately centre upon the total blood volume calculation.

The present invention envisages processing from about 0.25 to about 2 blood volumes by plasma exchange. In some embodiments, from about 0.25 to about 0.5 blood volumes may be subjected to plasma exchange. For example, from about 0.3 to about 0.4 blood volumes may be subjected to plasma exchange. In one embodiment, about 0.33 blood volumes may be subjected to plasma exchange.

In other embodiments, from about 0.75 to about 2 blood volumes may be subjected to plasma exchange. For example, from about 1 to about 2 blood volumes may be subjected to plasma exchange. Such as, from about 1 to about 1.5 blood volumes may be subjected to plasma exchange. In one embodiment, about 1 blood volume may be subjected to plasma exchange.

The skilled person will appreciate that aside from Nadler's formula other less utilised formulae and formulae centred around blood volume averages for a particular range of body weight can also be utilised to determine a patient's total blood volume. Such alternative methodologies are also within the scope of the present invention. For the purposes of the present invention, the relevant variable is blood volume regardless of the method utilised to calculate same. Minor variances in the quantum of blood volume arising from the use of different formulae will not have an effect on the efficacy of the present invention.

In one embodiment, the patient diagnosed with the cancer may have from about 10% to about 95% of their plasma removed from their blood as part of the plasma exchange procedure. In some embodiments, from about 10% to about 50% of their plasma may be removed from their blood. In other embodiments, from about 10% to about 40% of their plasma may be removed from their blood. For example, from about 20% to about 40% of their plasma may be removed from their blood. In yet other embodiments, from about 15% to about 30% of their plasma may be removed from their blood.

In further embodiments, from about 50% to about 95% of their plasma may be removed from their blood. In other embodiments, from about 60% to about 95% of their plasma may be removed from their blood. For example, from about 60% to about 90% of their plasma may be removed from their blood. For example, from about 60% to about 85% of their plasma may be removed from their blood. In yet other embodiments, from about 60% to about 80% of their plasma may be removed from their blood. In certain embodiments, from about 60% to about 75% of their plasma may be removed from their blood.

In some embodiments, the patient is subjected to multiple iterances of plasma exchange. For example, each iterance of plasma exchange may occur within 1 to 45 days of the previous iterance. In other embodiments, each iterance of plasma exchange may occur within 1 to 30 days of the previous iterance. For example, each iterance of plasma exchange may occur within 1 to 15 days of the previous iterance. In some embodiments, each iterance of plasma exchange may occur within 1 to 7 days of the previous iterance. In certain embodiments, each iterance of plasma exchange may occur within 1 to 3 days of the previous iterance.

It should be appreciated by those skilled in the art that the specific embodiments disclosed within paragraphs [0029]-[0037] should not be read in isolation, and that the present specification intends for these embodiments to be disclosed in combination with other embodiments as opposed to being disclosed individually. For example, each of the embodiments disclosed in paragraphs [0029]-[0037] is to be read as being explicitly combined with each of the embodiments in paragraphs [0009]-[0028], or any permutation of 2 or more of the embodiments disclosed therein.

### Replacement Fluids

Depending on the blood volume processed by the plasma exchange procedure (and as a result the percentage of plasma removed from the patient's blood) a patient may receive replacement fluids following a plasma exchange procedure to avoid hypotension and peripheral oedema. Typically, larger blood volumes require the administration of a replacement fluid to compensate for the volumes of plasma removed from the patient's blood. Suitable non-limiting examples of replacement fluids include albumin preparations, and fresh frozen plasma diluted with saline. Smaller blood/plasma volumes, such as those equivalent to plasma donation volumes do not usually require replacement fluids.

Between 70 and 80% of the oncotic activity in normal human plasma is attributable to its albumin content, which lies in the range of around 35-50 g/L. Consequently, the volume of albumin to be administered as a replacement fluid can be readily calculated based on the blood volume subjected to plasma exchange/the volume of plasma removed from the patient's blood.

Accordingly, in certain embodiments the patient may be administered between 10 g and 60 g of albumin per Litre of plasma removed by the plasma exchange procedure. For example, the patient may be administered between 30 g and 50 g of albumin per Litre of plasma removed by the plasma exchange procedure.

An iso-oncotic solution of human serum albumin is the most common choice for plasma replacement in plasma exchange. Naturally, this replacement fluid strategy may lead to a transient decline in the levels of non-albumin plasma constituents including coagulation factors, immunoglobulins, transport proteins, and complement components. In practice, redistribution and re-synthesis keep most other plasma proteins in a satisfactory range. In some circumstances, augmentation therapy with other plasma constituents may be performed should deficiencies arise.

It should be appreciated by those skilled in the art that the specific embodiments disclosed within paragraphs [0039]-[0042] should not be read in isolation, and that the present specification intends for these embodiments to be disclosed in combination with other embodiments as opposed to being disclosed individually. For example, each of the embodiments disclosed in paragraphs [0039]-[0042] is to be read as being explicitly combined with each of the embodiments in paragraphs [0009]-[0038], or any permutation of 2 or more of the embodiments disclosed therein.

### BRIEF DESCRIPTION OF THE DRAWINGS

Additional features and advantages of the present invention will be made clearer in the appended drawings, in which:

FIG. 1 plots the results of size exclusion chromatography (SEC) and flow cytometry analysis and identifies the presence of extracellular vesicles (EVs) in plasma after standard centrifugation, total plasma exchange (TPE) and low volume plasma exchange (LVPE);

FIG. 2 is a graph illustrating extracellular vesicle concentrations and sizes in isolated fractions as measured by nanoparticle tracking analysis;

FIG. 3 plots the relative amounts of EV-associated proteins including TSG101, Flotillin and Syntenin from plasma obtained by standard centrifugation, LVPE and TPE as detected by Western Blot;

FIG. 4 depicts the structure and size distribution of extracellular vesicles in plasma obtained by standard centrifugation, LVPE and TPE using CRYO-TEM; and

FIG. 5 shows Venn diagrams of protein EVs in plasma and PDXO. 5a) Venn diagrams of EV proteins from metastatic CRC KRAS mutant plasma samples; 5b) Venn diagrams of EV proteins identified in PDXO samples; and 5c) Venn diagram of common EV proteins from plasma and PDXO samples.

### DETAILED EXAMPLES OF THE INVENTION

It should be readily apparent to one of ordinary skill in the art that the examples disclosed herein below represent generalised examples only, and that other arrangements and methods capable of reproducing the invention are possible and are embraced by the present invention.

### Example 1—Assessing the Impact of Plasma Exchange on Extracellular Vesicle (EV) Depletion from Plasma

A study was performed to analyse the impact of plasma exchange on extracellular vesicle isolation/removal. In order to carry out the study, plasma was obtained from five blood donors (n=5) using standard centrifugation, and two different apheresis/plasmapheresis devices.

Blood was obtained from five different human donors. From each of the five donors, two whole blood bags (approximately 250 mL each) were filled to ensure homogenous sampling. Plasma samples were obtained from the donors' blood using standard centrifugation [A], the Spectra Optia® Apheresis System (Terumo BCT, Inc.) [B] and the Autopheresis-C™ Plasmapheresis System (Fresenius Kabi) [C]; further particulars of which are outlined below. Whilst both apheresis systems separate plasma from blood, they do so utilising different mechanical principals.

### Plasma Obtained by Standard Centrifugation

For donor 1, 15 mL of blood from each of blood bag I and blood bag 11 were pooled into a 50 mL Falcon tubes. The resulting 30 mL pool of human blood were then subjected to centrifugation for 10 min at 500×g followed by 15 minutes at 2,500×g using the Centrifuge 5702R (Eppendorf, Hamburg, Germany). This process was repeated for each of the remaining donors 2 to 5. On average, 14 to 16 mL of plasma were isolated from the blood samples, aliquoted in cryotubes and stored at −80° C. until evaluation.

### Plasma Obtained Using the Spectra Optia® Apheresis System (Terumo BCT, Inc.)

The Spectra Optia Apheresis System is an automatic and continuous blood component separator that uses centrifugation and optical detection to perform plasma separation procedures. It is generally used for large volume separations (>1 L) but can also be used with lower volume separations (<900 mL).

For donor 1, 200 mL of blood from blood bag I was processed using the Spectra Optia Apheresis System. This process was repeated for each of the remaining donors 2 to 5. On average, 70 to 110 mL of plasma were isolated from the blood samples, aliquoted in cryotubes and stored at −80° C. until evaluation.

### Plasma Obtained Using the Autopheresis-C™ Plasmapheresis System (Fresenius Kabi)

The Autopheresis-C system is a blood component separator that uses utilizes a spinning elongate membrane combined with a tangential filtration to mechanically separate plasma from blood. It is generally used for lower volume separations up to a maximum of 880 mL.

For donor 1, 200 mL of blood from blood bag 11 was processed using the Autopheresis-C system. This process was repeated for each of the remaining donors 2 to 5. On average, 50 to 80 mL of plasma were isolated from the blood samples, aliquoted in cryotubes and stored at −80° C. until evaluation.

### Isolation and Processing of EVs

To assess the impact of plasma exchange on extracellular vesicle isolation, extracellular vesicles from the obtained plasma samples were separated by size exclusion chromatography (SEC).

Two millilitres of sample (plasma) were used for isolating extracellular vesicles. Columns were manually loaded with 10 ml Sepharose™ CL-2B, and once tempered at room temperature, samples were carefully loaded onto the columns. Different fractions (approximately 35 fractions, 500 μL each) were progressively collected using PBS1× as elution buffer. All fractions were firstly characterized for their protein concentration using a Nanodrop® UV-Vis spectrophotometer (Abs 280 nm).

Low protein content fractions (namely, lacking albumin), as determined by Nanodrop® UV-Vis spectrophotometry, were analysed by flow cytometry to determine extracellular vesicle enriched fractions. EVs were detected by the presence of three classical extracellular vesicle markers: tetraspanins CD63, CD9 and CD81.

The four fractions showing the highest MFI fold change were pooled and identified as the extracellular vesicle-enriched fraction. Additionally, the four fractions with the highest protein concentration were pooled and identified as the protein-enriched fraction. Finally, the four fractions in-between the protein-enriched and extracellular vesicle-enriched fractions were pooled and identified as the intermediate-fraction. Then, total protein concentration from the different pooled fractions was determined by bicinchoninic acid assay (MicroBCA™ Protein Assay kit, ThermoFisher Scientific) following the manufacturer's instructions.

Flow cytometry with extracellular vesicle markers (vide supra) was used to identify extracellular vesicle enriched fractions, which were analysed for the presence of additional extracellular vesicle markers by western blot. Antibodies against TSG101 (ab30871, abcam), Flotilin-1 (ref: 610820, BD Biosciences) and Syntenin (ab133267, abcam) were used following a standard protocol.

From each of the three groups (standard centrifugation, Spectra Optia and Autopheresis-C) twenty micrograms from the extracellular vesicle-enriched pool was subjected to size distribution analysis and the estimated extracellular vesicle concentration was determined using Nanoparticle Tracking Analysis (NTA) (NanoSight NS300, Malvern Instruments Limited) following the manufacturer's instructions. Samples were diluted 1:40 with sterile and filtered PBS and, each sample was measured three times. The mean of the three replicates was considered as the final measurement. A volume of 3-5 μL of the different fractions (EV-enriched fraction, protein-enriched fraction and intermediate-fraction) were pooled for CRYO-TEM analysis. Size distribution quantification was done by calculating the diameter of all vesicles found in the images obtained by CRYO-TEM. At least, 50 vesicles were counted for each sample.

### Discussion of Results

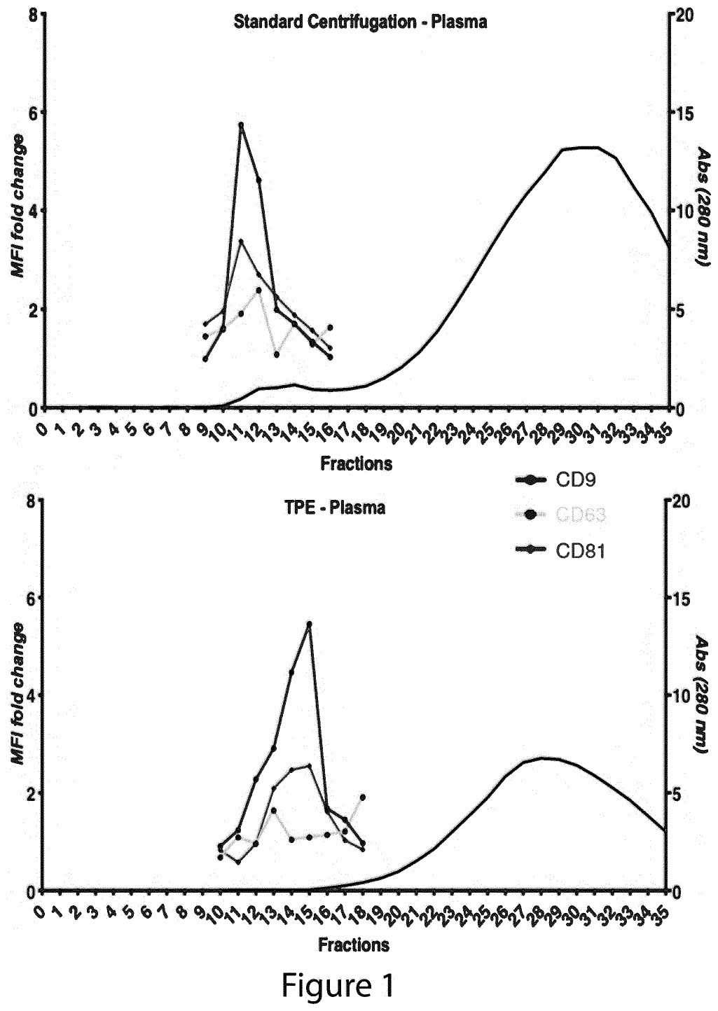

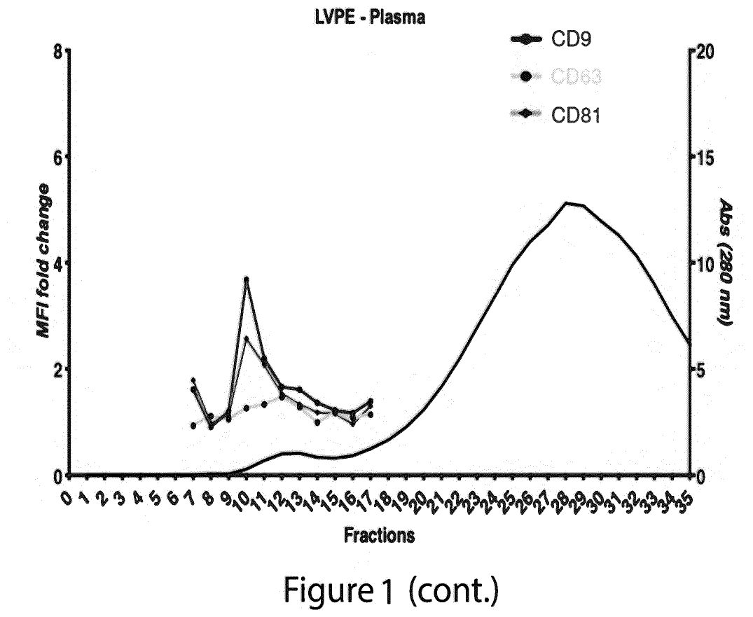

As shown in FIG. 1, SEC followed by flow cytometry facilitated the identification of extracellular vesicle enriched fractions in plasma obtained through each of the procedures disclosed in paras. [0058]-[0062]. Results from each of the plasmapheresis procedures are comparable to the fractions obtained from the standard centrifugation cohort.

FIG. 1 evidences the presence of tetraspanins CD81, CD9 and CD63 (which are representative extracellular vesicle biomarkers) in a similar sample range for each of the three groups (Centrifuge, Spectra Optia, and Autopheresis-C).

As disclosed in [0062] supra, nanoparticle tracking analysis was utilised to determine extracellular particle concentrations and size distribution in the extracellular vesicle enriched fractions from the donors' samples.

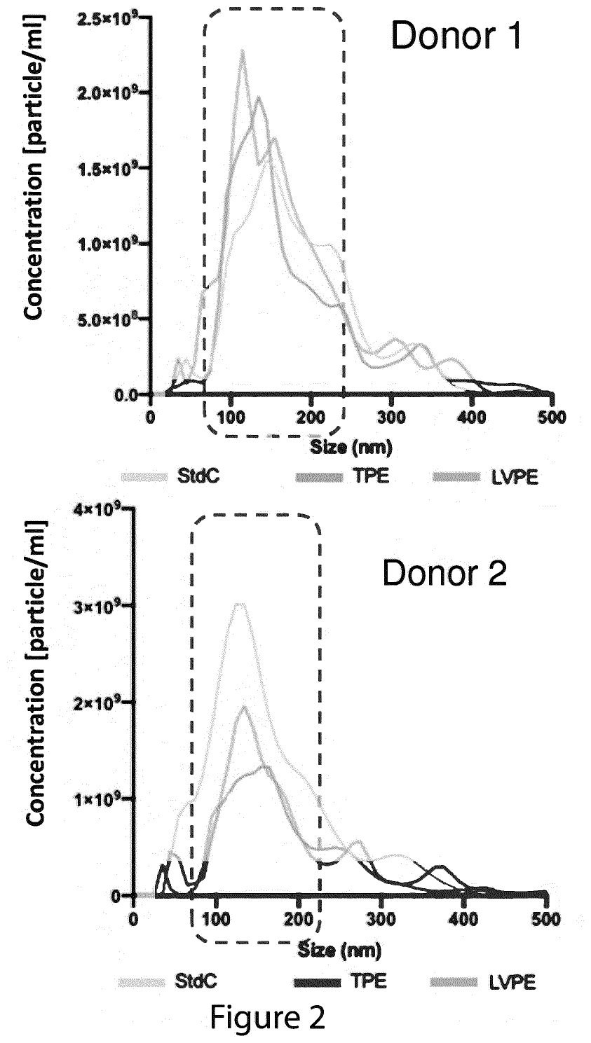

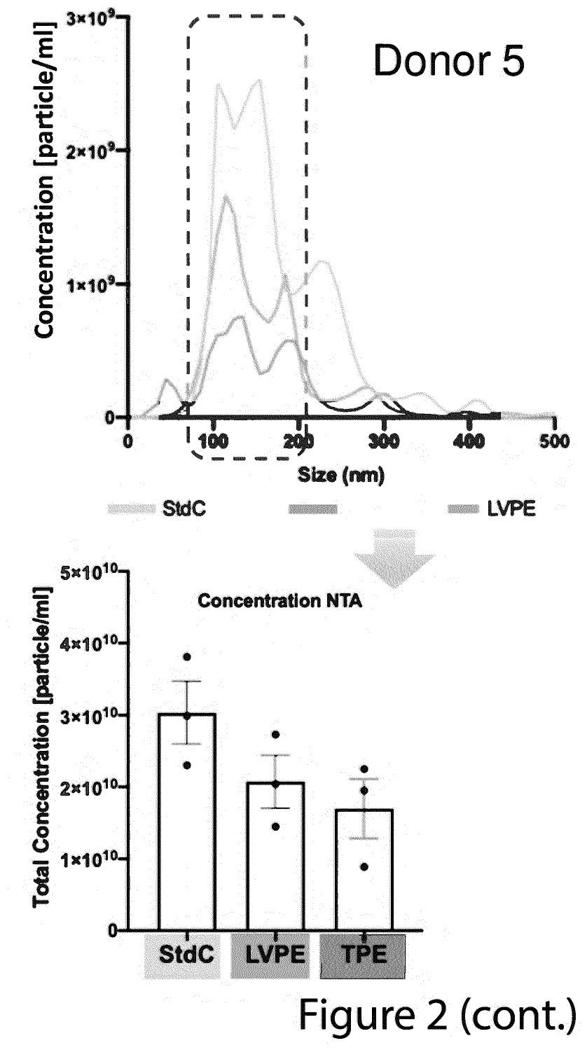

The plots in FIG. 2 reveal that extracellular vesicle enriched fractions have comparable particle concentration (particle/mL) and size distribution in plasma obtained by the different methodologies: standard centrifugation (“StdC”), Spectra Optia processing (“TPE”) and Autopheresis-C processing (“LVPE”) in the Figures respectively. The concentrations found were typically in the range of 10⁹-10¹² particles/mL.

As disclosed in [0061] supra, the molecular composition of the extracellular vesicles was determined by Western Blot for further confirmation that the isolated species were in fact extracellular vesicles. Samples were compared to a positive control provided by a SH-SY5Y cell line lysate known to contain the protein markers.

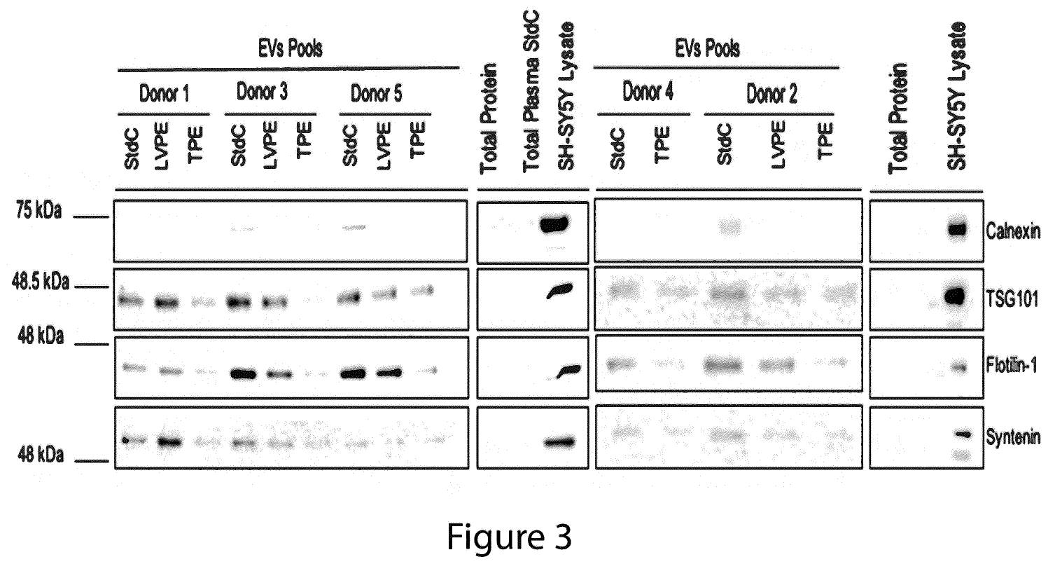

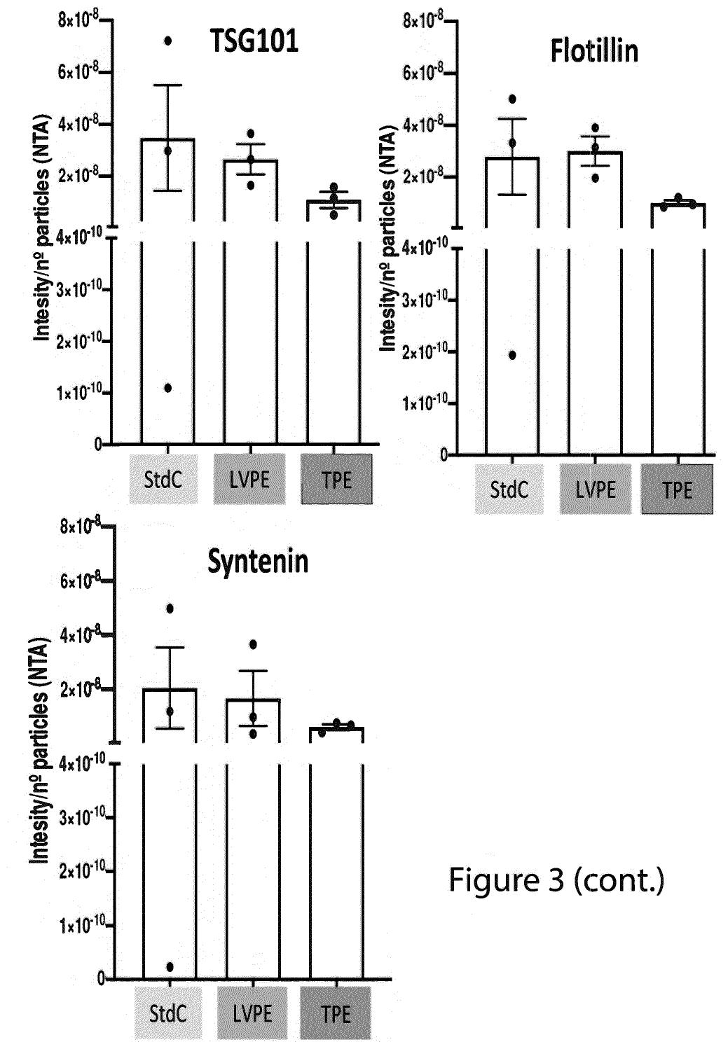

From FIG. 3 it is clearly demonstrated that the additional extracellular vesicle markers (TSG101, Flotilin-1, and Syntenin) were determined to be present by Western Blot in the extracellular vesicle enriched fractions from plasma obtained by standard centrifugation, LVPE and TPE. The relative intensity of each biomarker in each of the standard centrifugation, TPE and LVPE groups is plotted as a bar chart in FIG. 3. The amounts are consistent across all three of standard centrifugation, TPE, and LVPE groups.

The different SEC fractions (EV-enriched fraction, protein-enriched fraction and intermediate-fraction) were subsequently analysed by CRYO-TEM microscopy. Vitrified specimens were prepared by placing 3 μL of a sample on a Quantifoil 1.2/1.3 TEM grid, blotted to a thin film and plunged into liquidethane-N₂(I) in the Leica EM CPC cryowork station in the Centro Nacional de Biotecnologia (Madrid, Spain) and at Universitat Autonoma de Barcelona (UAB, Barcelona, Spain). The grids were transferred to a 626 Gatan cryoholder and maintained at −179° C. The grids were analyzed with a Jeol JEM 2011 transmission electron microscope operating at an accelerating voltage of 200 kV. Images were recorded on a Gatan Ultrascan 2000 cooled charge-coupled device (CCD) camera with the Digital Micrograph software package (Gatan). Size distribution quantification was done calculating the diameter of all vesicles found in the images. At least 50 vesicles were counted for each sample.

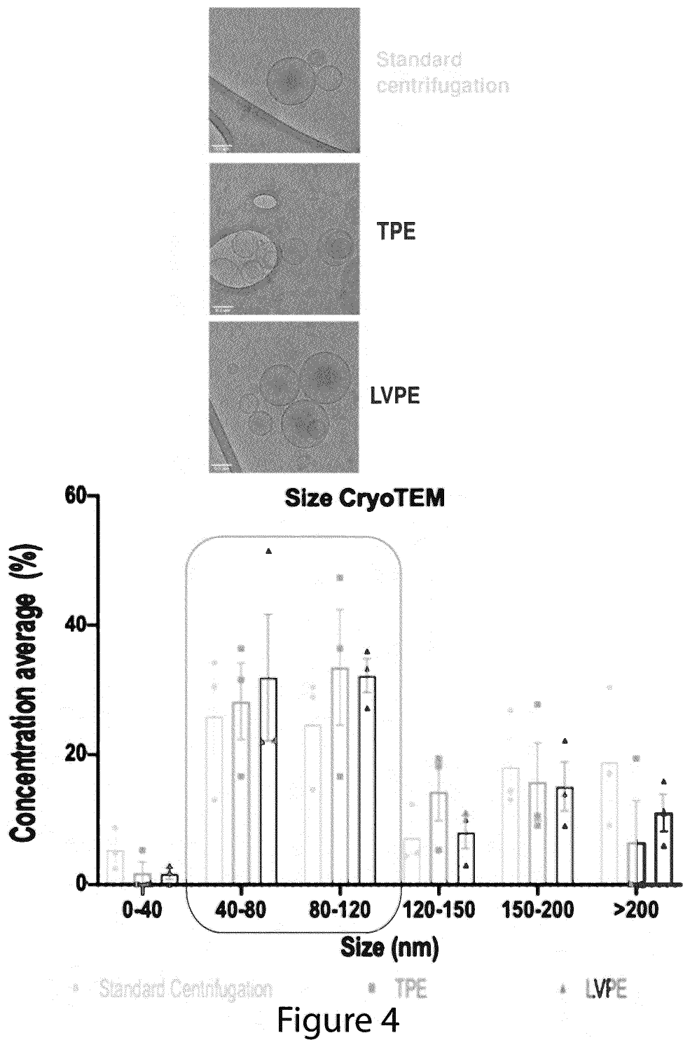

FIG. 4 illustrates the results from the CRYO-TEM analysis and demonstrates little variance in structure and size distribution of extracellular vesicles in plasma obtained by either standard centrifugation, LVPE or TPE. The majority of the extracellular vesicles were found to be in a size range of between 40 to 120 nm.

### Example 2—Identification of Proteins in EVs from Metastatic KRAS Mutant Colorectal Cancer Resistant to Chemotherapy

A proteomic analysis of EVs from plasma and xenografts samples from metastatic colorectal cancer (CRC) patients mutant for KRAS and resistant to first line chemotherapy was performed in order to identify potential tumor-specific EV proteins.

2 mL of plasma from three colorectal metastatic cancer patients mutant for KRAS (KRAS mut) and resistant to first line treatment were obtained from whole blood collected in tubes containing EDTA anticoagulant. Briefly, complete blood was centrifugated at 1500 g 10 minutes to eliminates cells. Platelet rich plasma fraction was collected and centrifugated at 2000 g 10 minutes 4° C. to eliminate platelets. Supernatants were again collected and stored at −80° C. until use. Before analysis, samples were thawed on ice, centrifugated at 12000 g 30 min 4° C. to eliminate debris and microparticles and filtered by 0.22 um (Sartorious).

Patient-derived xenografts (PDXs) were obtained from metastatic CRC patients mutant for KRAS and resistant to first line chemotherapeutics from Vall d'Hebron hospital (Barcelona, Spain). CM were obtained from patient-derived xenograft organoids (PDXOs) embedded in matrigel (Corning). Briefly, 150000 cells were mixed in a drop of 25 uL using a matrigel: culture media proportion of 1:1.5. Drops of cells were seeded and let form gel 30 min at 37° C. After that, 500 μL of culture media containing essential factors Noggin, R-spondin and Wnt3a was added to each well and plates were incubated at 37° C. 5% CO2. 48 hours later, CM were collected, and new media was added. While organoids were growing, CM was collected every other day. After reaching confluence, organoids were broken up from the Matrigel using cell recovery solution (Gibco), trypsinized and seeded again in the same conditions until the collection of the required CM volume (approx. 10-15 mL) was completed. CM were processed immediately after collection. Briefly, CM from these cultures was collected and centrifugated at 200 g 10 min to eliminate cells. Supernatant was centrifugated at 2000 g 10 min to eliminate debris. Finally, supernatant was centrifugated at 12000 g 30 min to eliminate microparticles, filtered by 0.22 um and stored at −80° C.

### Isolation of EVs

Size exclusion chromatography was performed using Izon geV2 commercial columns containing a pore size of 35 nm. 2 mL of sample (plasma or PDXOs CM) were loaded into the column. For 10-15 mL of PDXO CM, 10 KDa Amicon was used for concentrating samples. Columns were equilibrated with 80 mL 1×PBS before use. Samples were loaded into the column and eluted in PBS. Fractions corresponding to void volume (1 to 13 mL) were discarded. Fractions containing EVs were collected, pooled and stored at 4° C. The rest of the chromatographic fractions were discarded. Pooled fractions were loaded over a 20% sucrose cushion and ultra centrifugated in a SORVAL SW100+ ultracentrifuge (Thermofisher) at 100 000 g for 2 hours at 4° C. Pellets were resuspended in PBS.

### Proteomics Protocol

Samples were denatured with Laemmli buffer under reducing conditions, heated 10 minutes at 95° C. and loaded into 10% polyacrylamide gels. Electrophoresis was carried out at 50 V. Once the samples were entered into the resolving gel, electrophoresis was stopped, and gel was stained o/n with Coomassie blue. The next day, the band containing the EV protein sample was cut in 1 mm² pieces and transferred to Eppendorf tubes for digestion. Gel pieces containing denatured EV-proteins were washed with 50 mM ammonium bicarbonate (BA) in 50% ethanol. First dehydration process consisted on washing the gel 15 min with 100% ethanol. 10 mM DTT in 50 mM BA was used for residual disulfide bonds rupture. After that, samples were carbamidomethylated with 55 mM Iodoacetamide in 50 mM BA 30 minutes in the dark. Then, washes with 25 mM BA, 25 mM BA-50% Acetonitrile (Acn) and dehydration of the gel with 100% Acn were performed prior to protein digestion using 13 pg/uL trypsin. Digestion process was carried out o/n at 37° C. The next day samples were additionally dehydrated with 100% Acn and peptides were extracted from gel with 0.2% trifluoracetic acid 30 min at RT. Finally, samples were evaporated using SpeedVac (Thermofisher).

Lyophilized tryptic digests were resuspended in a solution containing 3% Acn and 0.1% fluoroacetic acid and analyzed using liquid Chromatography-Mass Spectrometry Analysis (LC-MS) using EVosep-One chromatograph and Orbitrap Eclipse Tribrid mass spectrometer (Thermo Fisher Scientific, Bremen, Germany). Xcalibur software package was used to control the mass spectrometer, version 4.2.28.14 (Thermo Fisher Scientific, Bremen, Germany). Peptide mixtures were separated by on-line nanoflow liquid chromatography using EVosep-One system (EVosep). Samples were first loaded on a trapping chromatographic column (Evotip C-18, EV-2001, Evosep) at 250 nL/min. Then, the peptide mixture was analyzed on the reversed-phase analytical column (EV1106 column: 150 um×150 mm, 1.9 um, Evosep). Elution of peptides was carried out using 0.1% formic acid in water (mobile phase A) and acetonitrile with 0.1% formic acid (mobile phase B), with a linear gradient from 0 to 35% of mobile phase B for 88 min at a flow rate of 250 nL/min. Ions were produced by applying a voltage of 2 kV to a stainless-steel nano-bore emitter (Proxeon, Thermo Fisher Scientific), attached to the end of the chromatographic column, on a Proxeon nano-spray flex ion source. Data-dependent mode was used to operate the Orbitrap Eclipse Tribrid mass spectrometer. A scan cycle started with a full-scan MS spectrum (from m/z 300 to 1600) obtained in the Orbitrap with setting a resolution of 30,000. The 20 most intense ions were targeted for collision-induced dissociation fragmentation in the linear ion trap when their intensity exceeded 1000 counts, dismissing singly charged ions. Collection of ions for both MS and MS/MS scans was achieved in the linear ion trap, and the AGC target values were set to 1×10⁶ ions for survey MS and 5000 ions for MS/MS scans. A maximum ion accumulation time was set to 500 and 200 ms in the MS and MS/MS modes, respectively. The normalized collision energy was set to 35%, and one microscan was obtained per each spectrum. Ions subjected to MS/MS with a relative mass window of 10 ppm were excluded from further sequencing for 20 s. A window of 20 ppm and isolation width of 2 Da was defined for all precursor masses. The lock mass option (m/z 445.120024) for survey scans was enabled in the Orbitrap measurements to improve mass accuracy.

Proteomic data were analyzed using the Proteome Discoverer v. 2.1 software (Thermo Fisher Scientific). Protein identification was performed using Mascot v. 2.5 (Matrix Science, London, UK) using the SwissProt database (2018_11, taxonomy limited to human proteins, 20,413 sequences). A precursor mass tolerance of 10 ppm was used to search the MS/MS spectra, fragment tolerance was set to 0.7 Da, trypsin specificity was controlled by setting a maximum of 2 missed cleavages, cysteine carbamidomethylation was set as fixed peptide modification, and methionine oxidation as variable peptide modification.

Protein identification files generated from Mascot (DAT files) were then loaded into the Scaffold software (version 3.00.07; Proteome software, Inc., Portland, OR, USA), resulting in a non-redundant list of identified proteins for each LC-MS/MS run per sample. Peptide identifications were validated whenever a PeptideProphet probability greater than 95% was achieved. Proteins identified with a probability higher than 95% and that contained at least two validated MS/MS spectra were accepted for further analysis. A false protein discovery rate (FDR) below 1.0%, as estimated by a database search, was achieved by using the described PeptideProphet and ProteinProphet tools. Using these filters, protein dataset files were generated by the “Scaffold software” using “spectral counts” (SpC) as a quantitative value.

### Bioinformatic Analysis

Samples were normalized by total counts. Dataset comparisons were performed using Venn diagrams (http://www.interactivenn.net/, DOI:https//doi.org/10.1186/s12859-015-0611-3).

### Results

Proteomic data analysis was performed including only proteins identified with at least two spectral counts across the dataset containing both EVs from plasma and PDXOs.

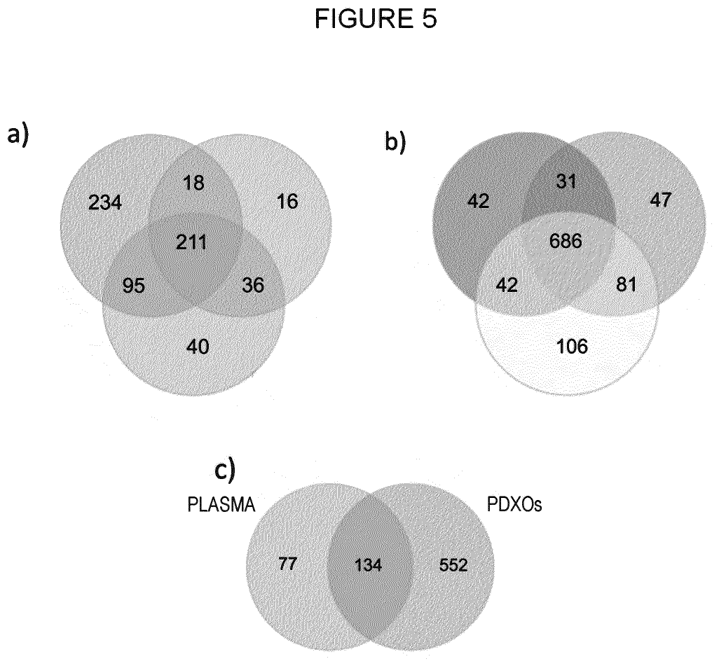

FIG. 5 shows the Venn diagrams of protein EVs in plasma and PDXOs. In FIG. 5a) Venn diagrams of EV proteins from three metastatic CRC KRAS mut plasma samples are shown. In FIG. 5b) Venn diagrams of EV proteins identified in three PDXOs are shown.

For plasma EVs, a non-redundant list of 650 proteins was identified out of the 558, 281 and 382 EV proteins identified from each patient. From those 650 EVs proteins, 211 proteins were common to all three patients (See FIG. 5a).

In PDXO-EVs samples, a non-redundant list of 1035 EV proteins was identified. A total of 686 proteins were shared by these three PDXO-EVs (FIG. 5b).

With the aim of identifying potential proteins associated to CRC, plasma and PDXO EVs proteins were compared to evaluate overlap. Proteins commonly present in all of three samples (plasma and PDXOs) were considered for this analysis. From 211 (plasma) and 686 (PDXO) EV proteins, 134 proteins were present in the two groups (FIG. 5c). From this, the most abundant plasma proteins which can be found in healthy samples, such as albumin, acute-phase proteins, apolipoproteins, immunoglobulins and keratins, were removed from the list. Finally, a total of 97 proteins was found to be present in both samples and thus, to be potentially associated to EVs from CRC patients.

These results herein presented show the identification of specific proteins in plasma-derived extracellular vesicles from tumor-derived plasma samples, in particular, from metastatic colorectal cancer (CRC) KRAS mutant plasma samples. In view of the results, when comparing with the PDXO samples, it can be inferred that these enriched proteins could clearly come from extracellular vesicles shed by colorectal tumor cells.

Consequently, the results herein shown suggest that by removing circulating EVs in cancer patients, EVs shed by tumors would also be removed. Thus, removal of EVs in cancer patients by plasma-exchange would represent an effective and reliable mean for cancer treatment.

1. Extracellular vesicle depleted human blood fractions for use in the treatment of cancer, wherein
blood obtained from a patient diagnosed with the cancer is subjected to plasma exchange so as to deplete the extracellular vesicle content prior to being re-administered to said patient.

2. The depleted blood fraction of claim 1, wherein from about 0.25 to about 2 blood volumes of the patient diagnosed with the cancer is subjected to plasma exchange.

3. The depleted blood fraction of any preceding claim, wherein from about 0.3 to about 0.4 blood volumes of the patient diagnosed with the cancer is subjected to plasma exchange.

4. The depleted blood fraction of any claims 1 to 2, wherein from about 1 to about 1.5 blood volumes of the patient diagnosed with the cancer is subjected to plasma exchange.

5. The depleted blood fraction of any claims 1 to 2, wherein the patient diagnosed with the cancer has from about 10% to about 95% of their plasma removed from their blood as part of the plasma exchange procedure.

6. The depleted blood fraction of claim 5, wherein the patient diagnosed with the cancer has from about 10% to about 40% of their plasma removed from their blood as part of the plasma exchange procedure.

7. The depleted blood fraction of claim 5, wherein the patient diagnosed with the cancer has from about 60% to about 95% of their plasma removed from their blood as part of the plasma exchange procedure.

8. The depleted blood fraction of any preceding claim, wherein the patient is subjected to multiple iterances of plasma exchange, each iterance of plasma exchange happening within 1 to 45 days of the previous iterance of plasma exchange.

9. The depleted blood fraction of claim 8, wherein each iterance of plasma exchange occurs within 1 to 7 days of the previous iterance of plasma exchange.

10. The depleted blood fraction of any preceding claim, wherein the cancer is selected from the group consisting of soft tissue sarcomas, kidney cancer, liver cancer, intestinal cancer, rectal cancer, leukaemia, lymphomas, brain cancer, oesophageal cancer, uterine cancer, cervical cancer, bone cancer, lung cancer, bladder cancer, breast cancer, laryngeal cancer, colorectal cancer, stomach cancer, ovarian cancer, pancreatic cancer, adrenal gland cancer, prostate cancer, and combinations thereof.

11. The depleted blood fraction of any preceding claim, wherein the cancer is a KRAS mutant cancer.

12. The depleted blood fraction of claim 11, wherein the KRAS mutant cancer is a cancer that is refractory to chemotherapy treatment.

13. The depleted blood fraction of claim 11 or 12, wherein the KRAS mutant cancer is a metastatic cancer.

14. Use of plasma exchange for removing extracellular vesicles from a patient in need thereof, wherein
the patient is a patient diagnosed with a cancer, and
further wherein no binding agent that specifically binds the extracellular vesicles is utilised in the plasma exchange process.

15. The use of claim 14, wherein from about 0.25 to about 2 blood volumes of the patient diagnosed with the cancer is subjected to plasma exchange.

16. The use of claims 14 to 15, wherein from about 0.3 to about 0.4 blood volumes of the patient diagnosed with the cancer is subjected to plasma exchange.

17. The use of claims 14 to 15, wherein from about 1 to about 1.5 blood volumes of the patient diagnosed with the cancer is subjected to plasma exchange.

18. The use of claims 14 to 15, wherein the patient diagnosed with the cancer has from about 10% to about 95% of their plasma removed from their blood as part of the plasma exchange procedure.

19. The use of claim 18, wherein the patient diagnosed with the cancer has from about 10% to about 40% of their plasma removed from their blood as part of the plasma exchange procedure.

20. The use of claim 18, wherein the patient diagnosed with the cancer has from about 60% to about 95% of their plasma removed from their blood as part of the plasma exchange procedure.

21. The use of claims 14 to 20, wherein the patient is subjected to multiple iterances of plasma exchange, each iterance of plasma exchange happening within 1 to 45 days of the previous iterance of plasma exchange.

22. The use of claim 21, wherein each iterance of plasma exchange occurs within 1 to 7 days of the previous iterance of plasma exchange.

23. The use of claims 14 to 22, wherein the cancer is selected from the group consisting of soft tissue sarcomas, kidney cancer, liver cancer, intestinal cancer, rectal cancer, leukaemia, lymphomas, brain cancer, oesophageal cancer, uterine cancer, cervical cancer, bone cancer, lung cancer, bladder cancer, breast cancer, laryngeal cancer, colorectal cancer, stomach cancer, ovarian cancer, pancreatic cancer, adrenal gland cancer, prostate cancer, and combinations thereof.

24. The use of claims 14 to 23, wherein the cancer is a KRAS mutant cancer.

25. The use of claim 24, wherein the KRAS mutant cancer is a cancer that is refractory to chemotherapy treatment.

26. The use of claim 24 or 25, wherein the KRAS mutant cancer is a metastatic cancer.
